(19) United States
(12) Patent Application Publication
(10) Pub. No.: US 2025/0345628 A1
(43) Pub. Date: Nov. 13, 2025

(54) PHOTOIRRADIATION PAD SUPPORT MEMBER, PHOTOIRRADIATION PAD SET, AND PHOTOTHERAPY APPARATUS

(71) Applicant: ATOM MEDICAL CORPORATION, Tokyo (JP)

(72) Inventor: Shinji ARAKI, Saitama City, Saitama (JP)

(21) Appl. No.: 18/865,405

(22) PCT Filed: Jun. 29, 2023
(86) PCT No.: PCT/JP2023/024263

§ 371 Date: Jul. 30, 2025

(30) Foreign Application Priority Data
Jun. 30, 2022 (JP) 2022-106357

(51) Int. Cl. A61N 5/06

(52) U.S. Cl. CPC A61N 5/0621

(57) ABSTRACT

Provided is a photoirradiation pad support member in which it is possible to suppress s bending of the photoirradiation pad toward a non-irradiation surface side while enabling skinship between a mother and a baby while continuing treatment for jaundice. A photoirradiation pad support member 1 is configured to support a photoirradiation pad in a state of being disposed on a non-irradiation surface side located on a reverse side from an irradiation surface of the photoirradiation pad, and is configured to suppress bending towards the non-irradiation surface side while allowing bending towards an irradiation surface side in a state of supporting the photoirradiation pad.

### TECHNICAL FIELD

The present invention relates to a photoirradiation pad support member, a photoirradiation pad set, and phototherapy apparatus. The present application claims priority based on Japanese Patent Application No. 2022-106357 filed on Jun. 30, 2022 in Japan, the content of which is incorporated herein by reference.

### BACKGROUND ART

In the related art, as phototherapy apparatus for treating neonatal jaundice, phototherapy apparatus that includes a flat plate-shaped photoirradiation pad is known (refer to, for example, PTL 1). According to the phototherapy apparatus of the related art, in a case where treatment light (hereinafter, may be simply referred to as “light”) is emitted from the photoirradiation pad toward a baby in a state where the baby is lying on the photoirradiation pad on the back, the back part of the baby can be uniformly irradiated with the light, and thus the treatment for jaundice can be performed more effectively. In addition, since the phototherapy apparatus is a so-called pad-type phototherapy apparatus, it is possible to have skinship between the mother and the baby, such as holding or breastfeeding the baby while continuing the treatment for jaundice.

### CITATION LIST

### Patent Literature

[PTL 1] JP-A-2013-248226

### SUMMARY OF INVENTION

### Technical Problem

By the way, in a field in which the pad-type phototherapy apparatus is handled, there is a demand that the photoirradiation pad is not excessively bent on the non-irradiation surface (surface on a reverse side from the surface on which light is emitted in the photoirradiation pad) side due to the structure of the photoirradiation pad. In response to this demand, for example, it is conceivable to increase the rigidity of the photoirradiation pad itself. However, in this case, not only the bending toward the non- irradiation surface side but also the bending toward the irradiation surface side is suppressed, and thus it becomes difficult to have skinship between the mother and the baby while continuing the treatment for jaundice.

The present invention has been made in view of the above circumstances, and an object of the present invention is to provide a photoirradiation pad support member, a photoirradiation pad set, and phototherapy apparatus, in which it is possible to suppress bending of the photoirradiation pad toward a non-irradiation surface side while enabling skinship between the mother and the baby while continuing treatment for jaundice.

### Solution to Problem

A photoirradiation pad support member according to the present invention is a support member that supports a photoirradiation pad that is used in phototherapy apparatus, in which the support member is configured to support the photoirradiation pad in a state of being disposed on a non- irradiation surface side located on a reverse side from an irradiation surface of the photoirradiation pad, and is configured to suppress bending towards the non-irradiation surface side while allowing bending towards an irradiation surface side, in a state of supporting the photoirradiation pad.

According to the present invention, the support member is configured to suppress bending toward the non-irradiation surface side while allowing bending toward the irradiation surface side in the state of supporting the photoirradiation pad. Therefore, by using the photoirradiation pad support member according to the present invention in the photoirradiation pad, it is possible to suppress the bending of the photoirradiation pad toward the non-irradiation surface side while making it possible to hold or breastfeed a baby while continuing the treatment for jaundice.

In the photoirradiation pad support member according to the present invention, preferably, the support member is formed in a plate shape and has a first surface having a cut with a predetermined depth and a second surface located on a reverse side from the first surface, and the support member is configured to support the photoirradiation pad in a state where the first surface faces the non-irradiation surface side and the second surface faces the irradiation surface side. That is, the support member is a plate-shaped member, the cut having a predetermined depth is provided on the first surface of the plate-shaped member, and the support member is configured to support the photoirradiation pad in a state where the first surface faces the non-irradiation surface side and the second surface faces the irradiation surface side. In this way, the photoirradiation pad support member according to the present invention, which exhibits an excellent effect, can be relatively easily realized.

In the photoirradiation pad support member according to the present invention, preferably, the support member has a structure in which the support member includes a plurality of plate pieces that are located on the first surface and a sheet-shaped base material layer that is located on the second surface and the plurality of plate pieces are arranged in a planar shape on the base material layer, and the cut is formed by a gap between the plate pieces adjacent to each other. In this case, since the photoirradiation pad support member according to the present invention can be manufactured relatively easily, for example, by bonding the plurality of plate pieces to the base material layer by using a pressure-sensitive adhesive, the manufacturing cost can be reduced.

In the photoirradiation pad support member according to the present invention, preferably, the support member has a single-layer structure in which a gap corresponding to the cut is formed on one surface of a plate material having a predetermined thickness. In this case, since the photoirradiation pad support member according to the present invention can be manufactured relatively easily, for example, by performing slitting on one surface of the plate material, the manufacturing cost can be reduced.

In the photoirradiation pad support member according to the present invention, preferably, a shape of the support member in a plan view is a substantially rectangular shape, and the cuts are formed along a longitudinal direction and a crosswise direction of the support member and along an oblique direction intersecting the longitudinal direction and the crosswise direction. In this way, since the photoirradiation pad support member can be more flexibly bent, the feeling of use (ease of bending when being bent toward the irradiation surface side) when holding or breastfeeding a baby by using the support member in the photoirradiation pad can be improved.

A photoirradiation pad set according to the present invention is a photoirradiation pad set made by combining a photoirradiation pad that is used in phototherapy apparatus, and the photoirradiation pad support member according to the present invention described above. Therefore, it is possible to suppress bending of the photoirradiation pad toward the non-irradiation surface side while making it possible to hold or breastfeed a baby while continuing treatment for jaundice.

Phototherapy apparatus according to the present invention is phototherapy apparatus including: a light source; a photoirradiation pad that emits light from the light source; a light guide part that guides the light from the light source to the photoirradiation pad; and the photoirradiation pad support member according to the present invention described above. Therefore, it is possible to suppress bending of the photoirradiation pad toward a non- irradiation surface side while making it possible to hold or breastfeed a baby while continuing treatment for jaundice.

### Advantageous Effects of Invention

According to the present invention, it is possible to suppress the bending of the photoirradiation pad toward the non-irradiation surface side while making it possible to hold or breastfeed a baby while continuing the treatment for jaundice.

### BRIEF DESCRIPTION OF DRAWINGS

FIG. 1 is a plan view of a photoirradiation pad support member 1 according to a first embodiment of the present invention.

FIG. 2 is a bottom view of the photoirradiation pad support member 1.

FIG. 3 is a front view of the photoirradiation pad support member 1 in a state where the photoirradiation pad support member 1 is flattened.

FIG. 4 is an enlarged view of a portion indicated by a reference numeral A1 in FIG. 3.

FIG. 5 is a diagram showing a state where the photoirradiation pad support member 1 is bent in the portion indicated by the reference numeral A1 in FIG. 3.

FIG. 6 is an enlarged view of a portion indicated by a reference numeral A2 in FIG. 1.

FIG. 7 is a diagram showing a state where the photoirradiation pad support member 1 is bent along a cut 12a.

FIG. 8 is a diagram showing a state where the photoirradiation pad support member 1 is bent along a cut 12b.

FIG. 9 is a diagram showing a state where the photoirradiation pad support member 1 is bent along a cut 12c.

FIG. 10 is an overall perspective view of phototherapy apparatus 100 according to the first embodiment of the present invention.

FIG. 11 is a diagram showing the inside of a photoirradiation pad 130.

FIG. 12 is a diagram schematically showing an optical fiber layer 132 disposed inside the photoirradiation pad 130.

FIG. 13 is a diagram for explaining a state where the photoirradiation pad 130 and the support member 1 are used.

FIG. 14 is a diagram for explaining a photoirradiation pad support member 2 according to a second embodiment.

FIG. 15 is a diagram for explaining phototherapy apparatus 300 according to a third embodiment.

### DESCRIPTION OF EMBODIMENTS

Hereinafter, embodiments of the present invention will be described with reference to the drawings. In the drawings, the length or thickness of each member is partially exaggerated for ease of understanding of the invention.

### Photoirradiation Pad Support Member according to First Embodiment

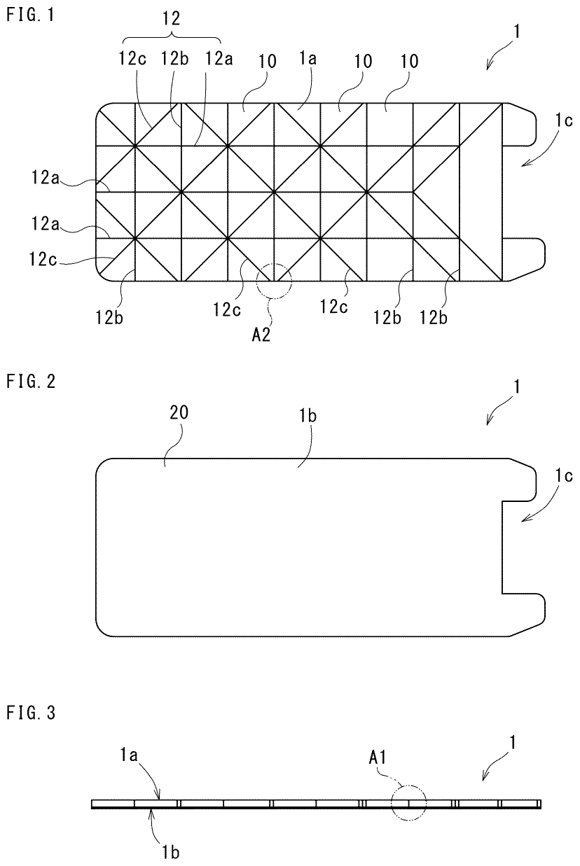

As shown in FIG. 1, a photoirradiation pad support member 1 (hereinafter, may be simply referred to as a “support member”) according to a first embodiment is a plate-shaped member having a substantially rectangular shape in a plan view. An upper surface (surface shown in FIG. 1) of the support member 1 is a first surface 1a, and a bottom surface (surface shown in FIG. 2) of the support member 1 is a second surface 1b. A cutout portion 1c that is cut out in a substantially rectangular shape in a plan view is provided on a right side (right side in FIGS. 1 to 3) of the support member 1.

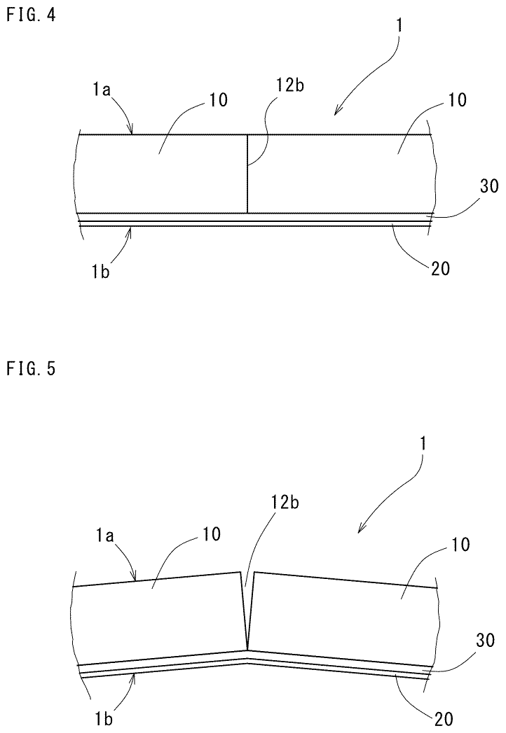

As shown in FIGS. 4 and 5, the support member 1 has a laminated structure in which a plurality of plate pieces 10 are arranged in a planar shape on a sheet-shaped base material layer 20, which is located on the second surface 1b side, with a pressure-sensitive adhesive layer 30 interposed therebetween.

Each of the plurality of plate pieces 10 is made of, for example, a plastic material such as a polypropylene resin. By using, for example, a low-foaming material (for example, a low-foaming plastic such as low-foaming polypropylene) as the plate piece 10, it is possible to reduce the weight of the entire support member while maintaining the required strength.

Each of the plurality of plate pieces 10 has a planar shape such as a triangle or a quadrangle, as shown in FIG. 1. The thickness of each plate piece 10 is the same, and is set to, for example, a thickness in a range of 1 mm to 20 mm.

The base material layer 20 is made of, for example, a vinyl-based multilayer sheet having excellent flexibility and durability. The thickness of the base material layer 20 is set to, for example, a thickness in a range of 0.1 mm to 2 mm.

The pressure-sensitive adhesive layer 30 is made of, for example, an acrylic double-sided pressure-sensitive adhesive tape or pressure-sensitive adhesive sheet. The thickness of the pressure-sensitive adhesive layer 30 is set to, for example, a thickness in a range of 1 mm or less.

As shown in FIGS. 1, 4, and 5, the first surface 1a of the support member 1 has cuts 12 (12a, 12b, and 12c) having a predetermined depth. The cut 12 is formed by a gap between the plate pieces 10 adjacent to each other, and the depth of the cut 12 is the same as the thickness of the plate piece 10. Each plate piece 10 is adhered to the pressure-sensitive adhesive layer 30. However, the plate pieces 10 adjacent to each other do not adhere to each other.

As shown in FIG. 1, the cuts 12 include a cut 12a formed along a longitudinal direction of the support member 1, a cut 12b formed along a crosswise direction of the support member 1, and a cut 12c formed along an oblique direction intersecting the longitudinal direction and the crosswise direction of the support member 1. The number of the cuts 12a is, for example, three, and the number of the cuts 12b is, for example, eight (excluding two cuts 12b provided in the vicinity of the cutout portion 1c), and the cuts 12a and the cuts 12b are arranged at regular intervals.

Since the planar shape of the support member 1 is a substantially rectangular shape, the cut 12a and the cut 12b are in an orthogonal relationship. The cut 12c is formed to intersect the cuts 12a and 12b at 45 degrees, and includes a plurality of cuts extending in an oblique direction from the upper right to the lower left of the drawing of FIG. 1 (as a slash symbol) and a plurality of cuts extending in an oblique direction from the upper left to the lower right of the drawing of FIG. 1 (as a backslash symbol).

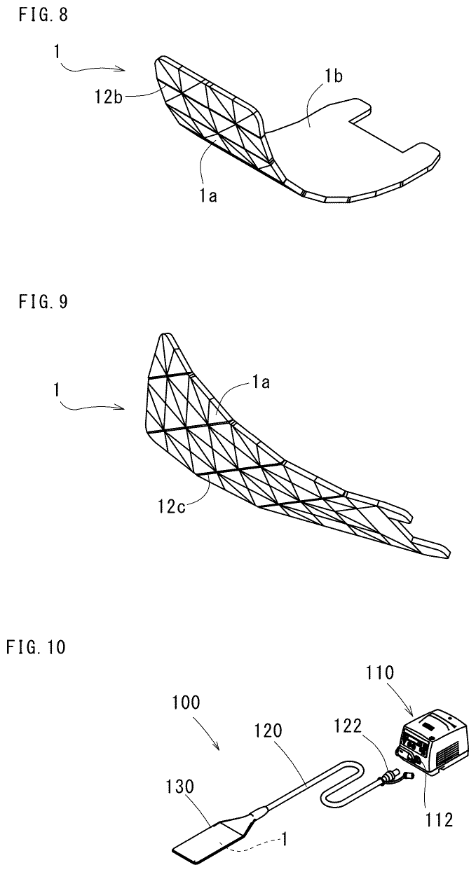

By forming the cuts 12a to 12c on the first surface 1a, as shown in FIGS. 7 to 9, the support member 1 can be bent toward the second surface 1b side with a relatively high degree of freedom. On the other hand, since cuts are not formed on the second surface 1b of the support member 1 (refer to FIG. 2), it is difficult to bend the support member 1 toward the first surface 1a side. That is, since the cuts 12a to 12c are formed on the first surface 1a, the support member 1 can be deformed such that the first surface 1a is convex and the second surface 1b is concave, and on the other hand, since the cuts are not formed on the second surface 1b of the support member 1, it is difficult to deform the support member 1 such that the first surface 1a is concave and the second surface 1b is convex.

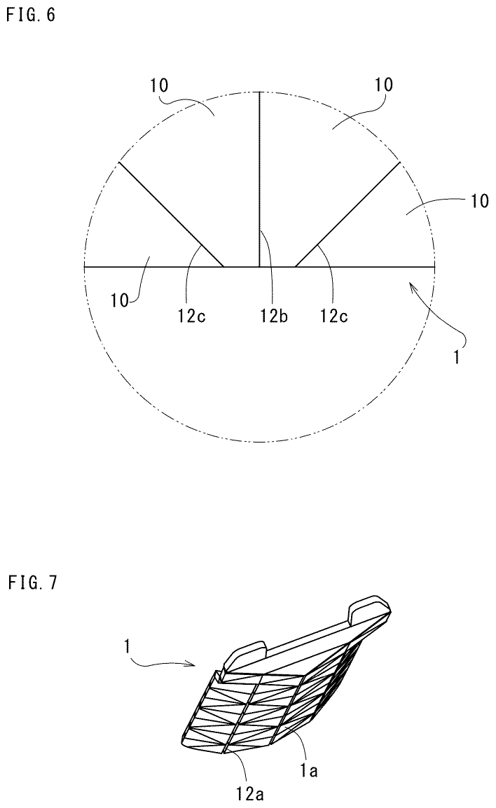

As shown in FIGS. 1 and 6, in a peripheral edge portion of the support member 1, the cut 12b formed along the crosswise direction of the support member 1 and the cut 12c extending in the oblique direction do not intersect with each other, and the cut 12c is configured such that the end portion of the cut 12c is located at a position separated from the end portion of the cut 12b by a predetermined distance. Also in the peripheral edge portion of the left side of the support member 1, similarly, the cut 12a formed along the longitudinal direction of the support member 1 and the cut 12c extending in the oblique direction do not intersect with each other, and the cut 12c is configured such that the end portion of the cut 12c is located at a position separated from the end portion of the cut 12a by a predetermined distance.

### Phototherapy Apparatus according to First Embodiment

As shown in FIG. 10, phototherapy apparatus 100 according to a first embodiment includes an apparatus main body 110 in which a light source (not shown) including a blue LED is incorporated, a light guide part 120 configured to be connectable to the apparatus main body 110, a photoirradiation pad 130 connected to the light guide part 120, and the support member 1 for supporting the photoirradiation pad 130. A combination of the photoirradiation pad 130 and the support member 1 is a photoirradiation pad set 150.

The phototherapy apparatus 100 is, for example, apparatus for performing phototherapy (also referred to as light therapy or light treatment) on a baby such as a newborn with jaundice symptoms. In the phototherapy apparatus 100 shown in FIG. 10, the support member 1 is disposed inside the photoirradiation pad 130.

The light guide part 120 has a structure in which, for example, a plurality of optical fibers are bundled in a rod shape, and has a function of guiding the light from the light source to the photoirradiation pad 130. A plug 122 is provided at one end portion of the light guide part 120 and is configured to be detachable from a connection socket 112 of the apparatus main body 110.

The photoirradiation pad 130 is, for example, a flat member having a substantially rectangular shape in a plan view, and is connected to and disposed at the other end portion of the light guide part 120. A plurality of optical fibers are disposed inside the photoirradiation pad 130. The optical fibers are disposed such that the optical fibers bundled in a rod shape are radially spread from the light guide part 120 side to the opposite side and are further arranged in a planar shape (refer to FIG. 12).

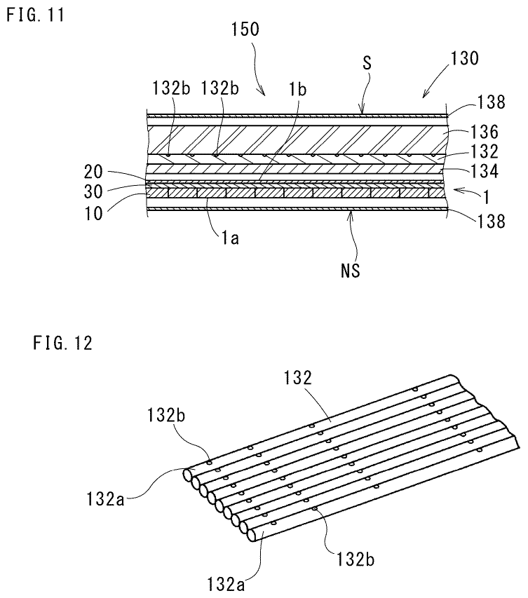

As shown in FIG. 11, the photoirradiation pad 130 includes an optical fiber layer 132, a reflective layer 134 that is disposed on a bottom side (lower side in FIG. 11) of the optical fiber layer 132, a protective layer 136 that is disposed on an upper side (upper side in FIG. 11) of the optical fiber layer 132, and a bag-shaped cover member 138. Three layers, the optical fiber layer 132, the reflective layer 134, and the protective layer 136, are laminated in a planar shape, and these three layers are covered with the bag-shaped cover member 138 from the outside (that is, the optical fiber layer 132, the reflective layer 134, and the protective layer 136 are disposed in the bag-shaped cover member 138).

The support member 1 is disposed between the reflective layer 134 and the cover member 138 (the lower cover member 138) in the bag-shaped cover member 138. Although the description by using illustration is omitted, the support member 1 that is disposed in the cover member 138 is disposed such that the cutout portion 1c of the support member 1 is located at the connection portion between the light guide part 120 and the photoirradiation pad 130 (that is, the cutout portion 1c faces the light guide part 120 side).

As shown in FIGS. 11 and 12, the optical fiber layer 132 is subjected to processing for emitting light toward the upper surface (irradiation surface S) side of the optical fiber layer 132. As the processing for emitting light, for example, processing disclosed in JP-A-2013-248226 (processing of forming a broken portion 132b by breaking a part of a clad layer in each optical fiber 132a by means such as laser cutting) or the like can be given.

The reflective layer 134 is, for example, a reflective sheet formed in a sheet shape, and is attached to the bottom side (a non-irradiation surface NS side) of the optical fiber layer 132 by using a double-sided tape or the like.

The protective layer 136 is, for example, a gel-like elastic body made of a synthetic resin, and has a function of protecting the optical fiber layer 132 by being disposed on the upper side (the irradiation surface S side) of the optical fiber layer 132. By using, for example, a urethane gel as the protective layer 136, high transparency and flexibility can be realized.

The cover member 138 is, for example, a member obtained by molding a sheet made of a synthetic resin in a bag shape. By using, for example, a urethane sheet as the cover member 138, high transparency and flexibility can be realized.

By using the photoirradiation pad 130 configured as described above, the light from the light source is emitted toward the upper side of FIG. 11. In this case, the upper surface of the photoirradiation pad 130 in FIG. 11 serves as the irradiation surface S, and the lower surface of the photoirradiation pad 130 in FIG. 11 serves as the non-irradiation surface NS. The support member 1 incorporated in the photoirradiation pad 130 is disposed such that the second surface 1b without the cut 12 faces the reflective layer 134.

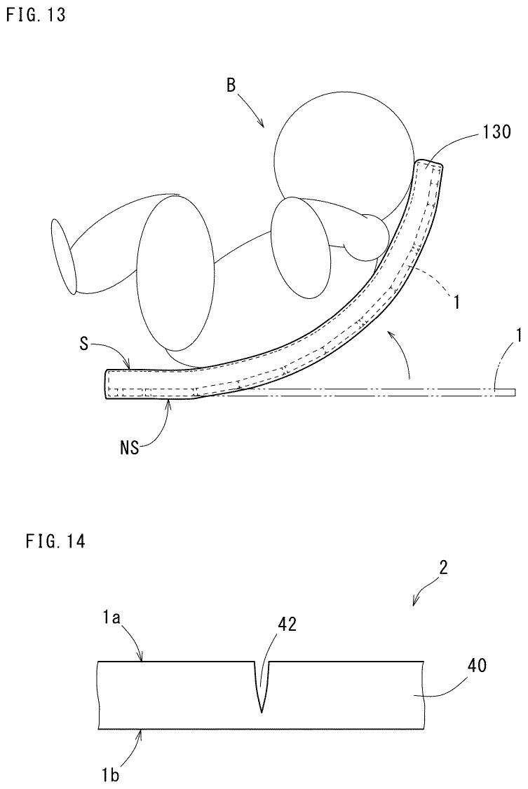

In a case where a baby B is placed on the irradiation surface S of the photoirradiation pad 130 in a state where the photoirradiation pad 130 is supported by the support member 1 according to the present embodiment, as shown in FIG. 13, the bending toward the irradiation surface S side is allowed, while the bending toward the non-irradiation surface NS side is suppressed.

That is, by using the support member 1 according to the present embodiment, it is possible to suppress the bending of the photoirradiation pad 130 toward the non-irradiation surface NS side while making it possible to hold or breastfeed the baby B while continuing the treatment for jaundice.

In the present embodiment, the support member 1 is configured to support the photoirradiation pad 130 in a state where the first surface la in which the cut 12 having a predetermined depth is formed faces the non-irradiation surface NS side and the second surface 1b faces the irradiation surface S side. In this way, it is possible to relatively easily realize the support member 1 that exhibits an excellent effect.

In the present embodiment, the support member 1 has a laminated structure in which the plurality of plate pieces 10 are arranged in a planar shape on the sheet-shaped base material layer 20 located on the second surface 1b side, and each of the cuts 12a to 12c is formed by a gap between the plate pieces 10 adjacent to each other. In this way, since the support member 1 can be manufactured relatively easily, it is possible to reduce the manufacturing cost.

In the present embodiment, since the cuts 12a to 12c described above are formed in the support member 1, the support member 1 can be more flexibly bent. As a result, it is possible to improve the feeling of use (ease of bending when being bent toward the irradiation surface S side) or the stability when holding or breastfeeding the baby B by using the support member 1 in the photoirradiation pad 130.

In the present embodiment, as shown in FIG. 6, a configuration is made such that the cut 12b and the cut 12c do not intersect each other at the peripheral edge portion of the support member 1 and the end portion of the cut 12c is located at a position separated from the end portion of the cut 12b by a predetermined distance. In other words, when the plate pieces 10 located on both sides of the cut 12b are viewed from a plane, the contour of each plate piece 10 is not a right-angled isosceles triangle but has a shape in which a corner portion located on the peripheral edge side of the support member 1 is chamfered (a triangle with a partially chamfered corner) among two acute angles constituting the right-angled isosceles triangle. According to the experiment conducted by the inventor of the present invention, it is found that in the case of a configuration made such that the end portion of the cut 12c is located at a position separated from the end portion of the cut 12b by a predetermined distance, as in the present embodiment, the bonding strength (peeling difficulty) between the plate piece 10 and the base material layer 20 when the support member 1 is bend toward the second surface 1b side can be increased, compared to a case where the end portion of the cut 12b and the end portion of the cut 12c are located at the same position (a case where the contour of each plate piece 10 located on both sides of the cut 12b is a right-angled isosceles triangle).

In the present embodiment, since the phototherapy apparatus 100 includes the support member 1 described above, as shown in FIG. 13, it is possible to suppress the bending of the photoirradiation pad 130 toward the non-irradiation surface NS side while making it possible to hold or breastfeed the baby while continuing the treatment for jaundice.

### Support Member according to Second Embodiment

FIG. 14 is a diagram for explaining the structure of a photoirradiation pad support member 2 (support member 2) according to a second embodiment, and corresponds to FIG. 4 in the first embodiment. The same components as those of the members described in the first embodiment are denoted by the same reference numerals, and the detailed description thereof will be omitted.

The support member 2 according to the second embodiment is different from the support member 1 according to the first embodiment in that the support member 2 has a single-layer structure (a plate material a having predetermined thickness) instead of a laminated structure.

A plate material 40 is a flexible plate-shaped member having a rectangular shape in a plan view and made of a plastic material, for example. The thickness of the plate material 40 is set to, for example, a thickness in a range of 1 mm to 20 mm.

As shown in FIG. 14, a gap 42 is formed on the first surface 1a of the plate material 40. The depth of the gap 42 is set to, for example, a depth in a range of 10% to 90% of the thickness of the plate material 40 (in a case where the thickness of the plate material is 10 mm, the depth of the gap 42 is in a range of 1 mm to 9 mm). As a method of manufacturing the plate material 40 in which the gap 42 is formed, for example, a known method such as slitting, laser processing, or press molding can be used.

The gap 42 corresponds to the “cut having a predetermined depth” in the present invention, and a plurality of gaps 42 are formed along the longitudinal direction, the crosswise direction, and the oblique direction of the support member 2, similarly to the cuts 12 described in the first embodiment. By forming the gaps 42 on the first surface 1a of the plate material 40, the support member 2 can be bent toward the second surface 1b side with a relatively high degree of freedom. On the other hand, since the gap 42 is not formed on the second surface 1b of the support member 2, it is difficult to bend the support member 2 toward the first surface 1a side. That is, since the gaps 42 are formed on the first surface 1a of the plate material 40, the support member 2 can be deformed such that the first surface 1a is convex and the second surface 1b is concave, and on the other hand, since the gap 42 is not formed on the second surface 1b of the support member 2, it is difficult to deform the support member 2 such that the first surface 1a is concave and the second surface 1b is convex.

As described above, the support member 2 according to the second embodiment is different from the support member 1 according to the first embodiment in that the support member 2 has a single-layer structure instead of a laminated structure. However, similarly to the support member 1 according to the first embodiment, when a baby is placed on the irradiation surface of the photoirradiation pad in a state where the photoirradiation pad is supported by the support member 2, the bending toward the irradiation surface side is allowed, while the bending toward the non-irradiation surface side is suppressed. That is, by using the support member 2 according to the present embodiment, it is possible to suppress the bending of the photoirradiation pad toward the non-irradiation surface side while making it possible to hold or breastfeed the baby while continuing the treatment for jaundice.

The support member 2 according to the second embodiment has the same configuration as the support member 1 according to the first embodiment except that the support member 2 has a single-layer structure instead of a laminated structure, and therefore, the support member 2 has a corresponding effect as it is, among the effects of the support member 1 according to the first embodiment.

### Phototherapy Apparatus according to Third Embodiment

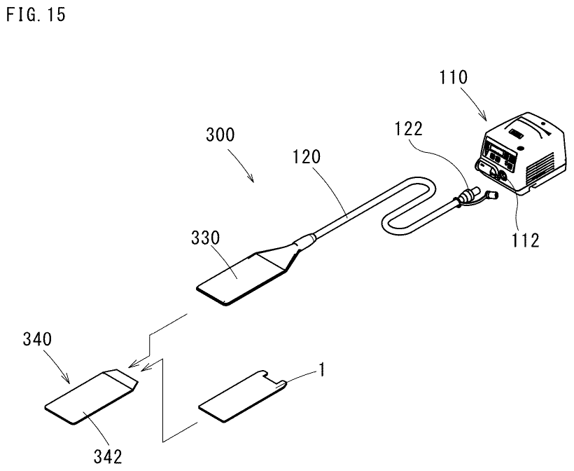

FIG. 15 is an overall perspective view of phototherapy apparatus 300 according to a third embodiment. The same components as those of the members described in the first embodiment are denoted by the same reference numerals, and the detailed description thereof will be omitted.

In the first embodiment, the support member 1 is incorporated in the photoirradiation pad 130 (refer to FIGS. 10 and 11). Whereas, in the third embodiment, the support member 1 is not incorporated in a photoirradiation pad 330 and the photoirradiation pad 330 and the support member 1 are stored in a storage body 340.

Although the description by using illustration of the photoirradiation pad 330 of the third embodiment is omitted, the photoirradiation pad 330 includes an optical fiber layer, a reflective layer, a protective layer, and a cover member, as in the photoirradiation pad 130 of the first embodiment. However, the support member 1 is not disposed in the cover member.

The storage body 340 is a flat member having, for example, a substantially rectangular shape in a plan view, similarly to the photoirradiation pad 330, and has a storage portion 342 formed in a pocket shape. The storage portion 342 is formed to have a size in which the photoirradiation pad 330 and the support member 1 can be stored. For example, a synthetic resin such as urethane is used for the storage body 340, and in a state where the photoirradiation pad 330 and the support member 1 are stored in the storage portion 342, light can be emitted toward a baby through the storage body 340.

In the phototherapy apparatus 300 according to the third embodiment, similarly to the phototherapy apparatus 100 according to the first embodiment, when a baby is placed on the irradiation surface (an upper surface of the storage portion 342) of the photoirradiation pad 330 in a state where the photoirradiation pad 330 is supported by the support member 1, the bending toward the irradiation surface side (upward the storage body 340) is allowed, while the bending toward the non-irradiation surface side (downward the storage body 340) is suppressed. That is, also in the case of the phototherapy apparatus 300 according to the third embodiment, similarly, it is possible to suppress the bending of the photoirradiation pad toward the non-irradiation surface side while making it possible to hold or breastfeed the baby while continuing the treatment for jaundice.

In the present embodiment, since the photoirradiation pad 330 and the support member 1 are stored in the storage portion 342 of the storage body 340, the support member 1 can be relatively easily taken out from the storage body 340 (the storage portion 342). In this way, for example, in a case where only the support member 1 is desired to be replaced, the present embodiment is convenient.

The phototherapy apparatus 300 according to the third embodiment is different from the phototherapy apparatus 100 according to the first embodiment in that the position of the support member 1 (whether or not the support member 1 is incorporated in the photoirradiation pad) or the like is different. However, the phototherapy apparatus 300 has the same configuration as the phototherapy apparatus 100 according to the first embodiment in other respects, and therefore, the phototherapy apparatus 300 has a corresponding effect as it is, among the effects of the phototherapy apparatus 100 according to the first embodiment.

The present invention is not limited to the embodiments described above, and various modifications can be made within a scope which does not depart from the gist of the present invention, and for example, the following modifications can also be made.

In the embodiments described above, a case where each of the plurality of plate pieces constituting the support member is made of a plastic material such as a polypropylene resin has been described as an example. However, there is no limitation thereto. In a case where the condition such as the required strength or weight is satisfied, for example, other materials such as metal can also be used.

In the embodiments described above, a case where a vinyl-based multilayer sheet is used as the base material layer constituting the support member has been described as an example. However, there is no limitation thereto. In a case where the conditions of flexibility, durability, and the like are satisfied, sheets or the like made of other materials may be used. In addition, by mixing fibers into a part of a resin material, the extension in an in-plane direction of the base material layer can be suppressed.

In the embodiments described above, a case where an acrylic double-sided pressure-sensitive adhesive tape or a pressure-sensitive adhesive sheet is used as the pressure-sensitive adhesive layer constituting the support member has been described as an example. However, there is no limitation thereto, and pressure-sensitive adhesive tapes or the like made of other materials may be used. In addition, in a case where a plurality of plate pieces can be attached to the base material layer, other known bonding means such as applying an adhesive may be used.

In the embodiments described above, a case where a synthetic resin such as urethane is used as the materials of the cover member and the storage body constituting the photoirradiation pad has been described as an example. However, there is no limitation thereto, and for example, other materials such as silicone rubber having transparency may be used.

In the embodiments described above, a case where the light source is disposed in the apparatus main body of the phototherapy apparatus and the light from the light source is emitted from the photoirradiation pad through the optical fiber of the light guide part has been described as an example. However, there is no limitation thereto. For example, the present invention can also be applied to a photoirradiation pad in which a plurality of LEDs are incorporated in the photoirradiation pad itself.

In the embodiments described above, a case where both of the planar shape of the photoirradiation pad and the planar shape of the support member are substantially rectangular has been described as an example. However, there is no limitation thereto, and other shapes such as a square, for example, may be used. In addition, the support member does not necessarily need to be provided with the cutout portion.

In the embodiments described above, a case where the cuts are formed along the longitudinal direction, the crosswise direction, and the oblique direction of the support member has been described as an example. However, there is no limitation thereto. For example, the cuts may be formed only along the longitudinal direction and the crosswise direction of the support member, or the cut may be formed only along the oblique direction of the support member. In a case where the planar shape of the support member is a shape other than a substantially rectangular shape, the direction of the cut can also be appropriately selected. In addition, the cut is not limited to a straight line in a plan view, and may be a curved line.

In the embodiments described above, a case where the support member is incorporated in the photoirradiation pad or is stored in the storage portion of the storage body has been described as an example. However, there is no limitation thereto. For example, the support member may be attached to the outer surface of the photoirradiation pad by using a hook-and-loop fasteners or the like. In addition, for example, in a case where the support member is transparent and does not block the light emitted from the photoirradiation pad, the support member can also be used by being attached to the irradiation surface of the photoirradiation pad. In this case, although there is a possibility that the skin of a baby directly touches the support member, since the cut is not formed in the contact surface of the support member (the surface in which the cut is formed faces the photoirradiation pad), there is no concern that the cut of the support member may directly touch the baby.

In the embodiments described above, the phototherapy apparatus for a baby such as a newborn has been described. However, there is no limitation thereto, and the present invention can be applied to phototherapy apparatus that is used for a patient other than the newborn. In addition, the type of disease to be treated with the phototherapy apparatus according to the present invention is not limited to the neonatal jaundice.

The material, shape, dimensions, number, disposition location, and the like of each component in the embodiments described above are optional and not limited as long as the present invention can be achieved.

### INDUSTRIAL APPLICABILITY

It is possible to suppress the bending of the photoirradiation pad toward the non-irradiation surface side while making it possible to hold or breastfeed a baby while continuing the treatment for jaundice.

### REFERENCE SIGNS LIST

1, 2: photoirradiation pad support member (support member)
1a: first surface (of support member)
1b: second surface (of support member)
10: plate piece
12, 12a to 12c: cut
20: base material layer
30: pressure-sensitive adhesive layer
40: plate material
42: gap (cut)
100, 300: phototherapy apparatus
110: apparatus main body
120: light guide part
130, 330: photoirradiation pad
150: photoirradiation pad set
B: baby
NS: non-irradiation surface (of photoirradiation pad)
S: irradiation surface (of photoirradiation pad)

1. A photoirradiation pad support member that is a support member that supports a photoirradiation pad that is used in phototherapy apparatus,
wherein the support member is configured to support the photoirradiation pad in a state of being disposed on a non-irradiation surface side located on a reverse side from an irradiation surface of the photoirradiation pad, and
is configured to suppress bending towards the non-irradiation surface side while allowing bending towards an irradiation surface side, in a state of supporting the photoirradiation pad.

2. The photoirradiation pad support member according to claim 1, wherein the support member is formed in a plate shape and has a first surface having a cut with a predetermined depth and a second surface located on a reverse side from the first surface, and
the support member is configured to support the photoirradiation pad in a state where the first surface faces the non-irradiation surface side and the second surface faces the irradiation surface side.

3. The photoirradiation pad support member according to claim 2, wherein the support member has a structure in which the support member includes a plurality of plate pieces that are located on the first surface and a sheet-shaped base material layer that is located on the second surface and the plurality of plate pieces are arranged in a planar shape on the base material layer, and
the cut is formed by a gap between the plate pieces adjacent to each other.

4. The photoirradiation pad support member according to claim 2, wherein the support member has a single-layer structure in which a gap corresponding to the cut is formed on one surface of a plate material having a predetermined thickness.

5. The photoirradiation pad support member according to claim 2, wherein a shape of the support member in a plan view is a substantially rectangular shape, and
the cuts are formed along a longitudinal direction and a crosswise direction of the support member and along an oblique direction intersecting the longitudinal direction and the crosswise direction.

6. A photoirradiation pad set made by combining a photoirradiation pad that is used in phototherapy apparatus and the photoirradiation pad support member according to claims 1.

7. A phototherapy Phototherapy apparatus comprising:
a light source;
a photoirradiation pad that emits light from the light source;
a light guide part that guides the light from the light source to the photoirradiation pad; and
the photoirradiation pad support member according to claim 1.

8. A photoirradiation pad set made by combining a photoirradiation pad that is used in phototherapy apparatus and the photoirradiation pad support member according to claim 2.

9. A photoirradiation pad set made by combining a photoirradiation pad that is used in phototherapy apparatus and the photoirradiation pad support member according to claim 3.

10. A photoirradiation pad set made by combining a photoirradiation pad that is used in phototherapy apparatus and the photoirradiation pad support member according to claim 4.

11. A photoirradiation pad set made by combining a photoirradiation pad that is used in phototherapy apparatus and the photoirradiation pad support member according to claim 5.

12. A phototherapy apparatus comprising:
a light source;
a photoirradiation pad that emits light from the light source;
a light guide part that guides the light from the light source to the photoirradiation pad; and
the photoirradiation pad support member according to claim 2.

13. A phototherapy apparatus comprising:
a light source;
a photoirradiation pad that emits light from the light source;
a light guide part that guides the light from the light source to the photoirradiation pad; and
the photoirradiation pad support member according to claim 3.

14. A phototherapy apparatus comprising:
a light source;
a photoirradiation pad that emits light from the light source;
a light guide part that guides the light from the light source to the photoirradiation pad; and
the photoirradiation pad support member according to claim 4.

15. A phototherapy apparatus comprising:
a light source;
a photoirradiation pad that emits light from the light source;
a light guide part that guides the light from the light source to the photoirradiation pad; and
the photoirradiation pad support member according to claim 5.
